(19) United States
(12) Patent Application Publication
(10) Pub. No.: US 2018/0161179 A1
(43) Pub. Date: Jun. 14, 2018

(54) Prosthetic Pylon Attachment Device

(71) Applicant: Ricky Eugene Sevier, Catoosa, OK (US)

(72) Inventor: Ricky Eugene Sevier, Catoosa, OK (US)

(21) Appl. No.: 15/373,185

(22) Filed: Dec. 8, 2016

(51) Int. Cl. A61F 2/60

(52) U.S. Cl. CPC A61F 2/60; A61F 2002/5001

(57) ABSTRACT

A prosthetic pylon attachment device is an apparatus for mechanically attaching protective and ornamental covers, and accessories, to prosthetic pylons. This function is provided by a semi-circular clip that has the same internal diameter as the outer diameter of the pylon, with a semicircular opening in the flexible clip allowing it to be securely installed onto the pylon with minimal force and without tools. It can also be easily removed without hand tools. A semicircular retaining clip is used to secure the clip in place and a braking pad is used to restrict rotation. Radial attachment receptors are place at intervals around the semicircular clip and on the rear attachment piece. Bridge pieces are snapped into the receptors and mounting pieces are snapped into the bridge pieces. These assemblies can be used in quantities and lengths as needed. The device can be easily reconfigured to use various attachments and accessories.

### RELATED APPLICATIONS

None.

### FEDERALLY SPONSORED RESEARCH OR DEVELOPMENT None.

### BACKGROUND

A typical lower extremity prosthesis typically consists of a socket that interfaces with the amputees' residual limb, attached to a tubular pylon that terminates at a prosthetic foot. In most cases, this pylon is left uncovered and unprotected. In some cases, an ornamental covering is wrapped around the socket, extending to the prosthetic foot, and either strapped, or fastened with bolts, to the prosthesis.

### SUMMARY

The primary object of the present invention is to provide a device to attach protective and ornamental covers, and accessories, to prosthetic pylons.

It is another object of the invention to provide an attachment device that can be easily removed without hand tools.

It is still another object of the invention to provide an attachment device that can be easily reconfigured to use many attachments and accessories.

These and other objects of the invention are provided by an extruded, semi-circular clip that has the same internal diameter as the outer diameter of the pylon. A semicircular opening in the flexible clip allows it to be installed onto the pylon with minimal force. Another piece the shape of the semicircular opening is used to secure the clip in place. Attachment receptors are placed at intervals around the semicircular clip and on the rear attachment piece. Bridge pieces are snapped into the receptors. Mounting pieces are snapped into the bridge pieces. These assemblies can be used in quantities as needed.

### BRIEF DESCRIPTION OF THE DRAWINGS

FIG. 1 is a side elevational view of a typical prosthetic pylon and foot attached to below the knee socket.

FIG. 2 is a front perspective view of an assembled embodiment of the invention installed on a prosthetic pylon.

FIG. 3 is a rear perspective view of an assembled embodiment of the invention installed on a prosthetic pylon.

FIG. 4 is a section view of the central and rear retaining portions of the invention indicating connector features.

FIG. 5 is a section view of the bridge and end clip portions of the invention indicating connector features.

FIG. 6 is a front perspective view of an embodiment of two of the invention components indicating the method of connection.

FIG. 7 is a rear perspective view of an embodiment of three of the invention components indicating the method of connection.

FIG. 8 is a front perspective view of an embodiment of two complete installed assemblies of the invention on an existing pylon.

### DETAILED DESCRIPTION OF THE INVENTION

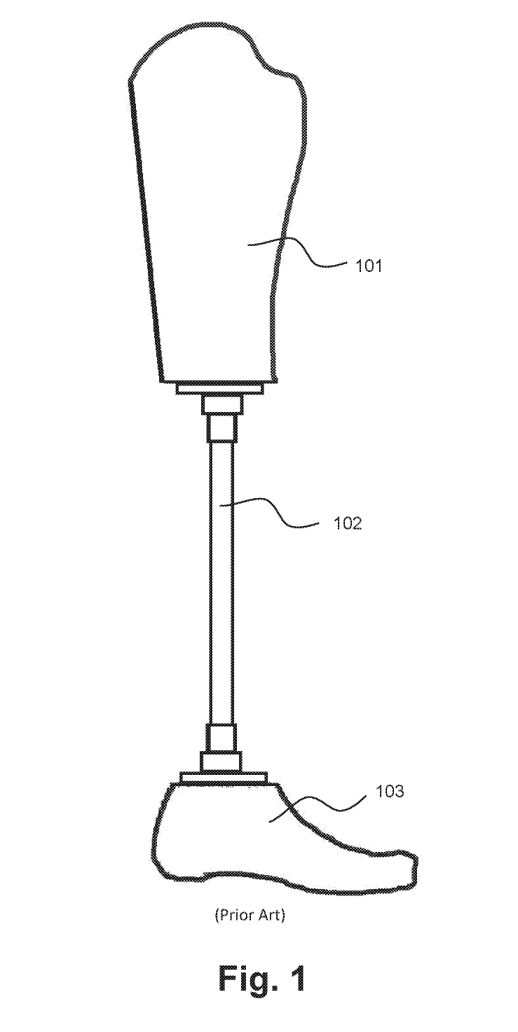

As shown in FIG. 1, a typical prosthetic device consists of a socket 101, a pylon 102, and a prosthetic foot 103. The socket 101 fits over the lower end of the amputees' residual limb. The pylon 102 attaches to a fitting on the lower end of the socket 101, and the prosthetic foot 103 is bolted to the lower end of the pylon 102.

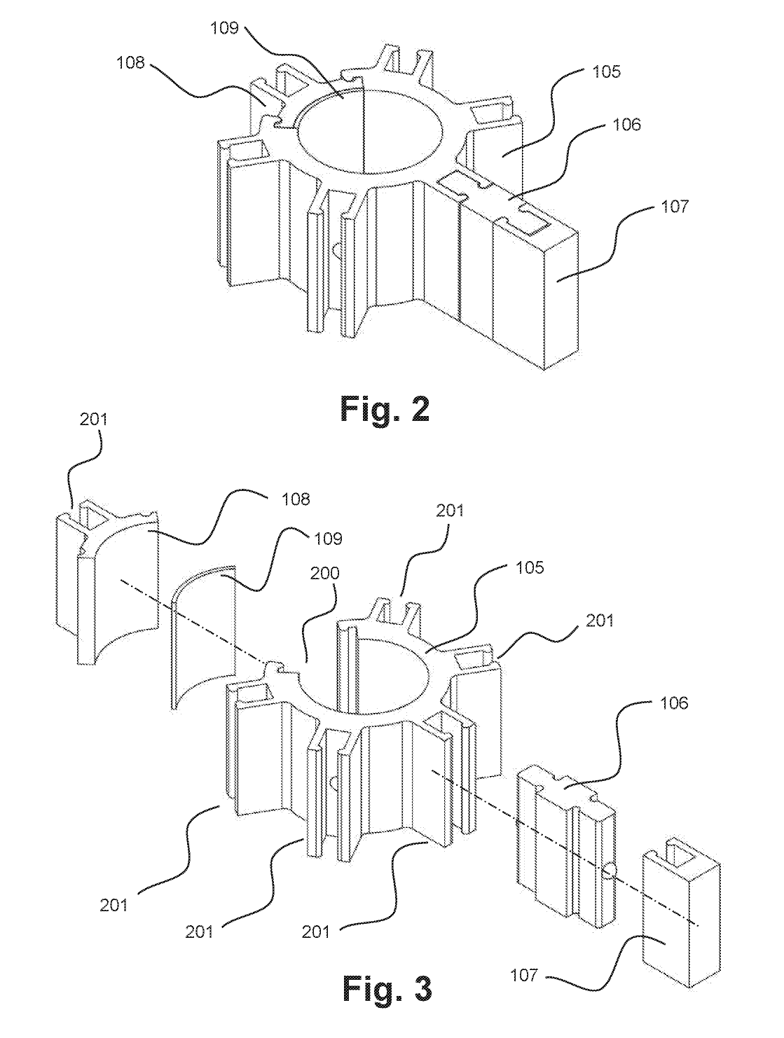

FIG. 2 shows the assembled embodiment of the present invention consisting of its 5 component part types; semi-circular clip 105, rear retaining clip 108, rotational braking pad 109, bridge 106 and end clip 107.

FIG. 3 shows the disassembled embodiment of the present invention consisting of its 5 component part types; semi-circular clip 105, rear retaining clip 108, rotational braking pad 109, bridge 106 and end clip 107.

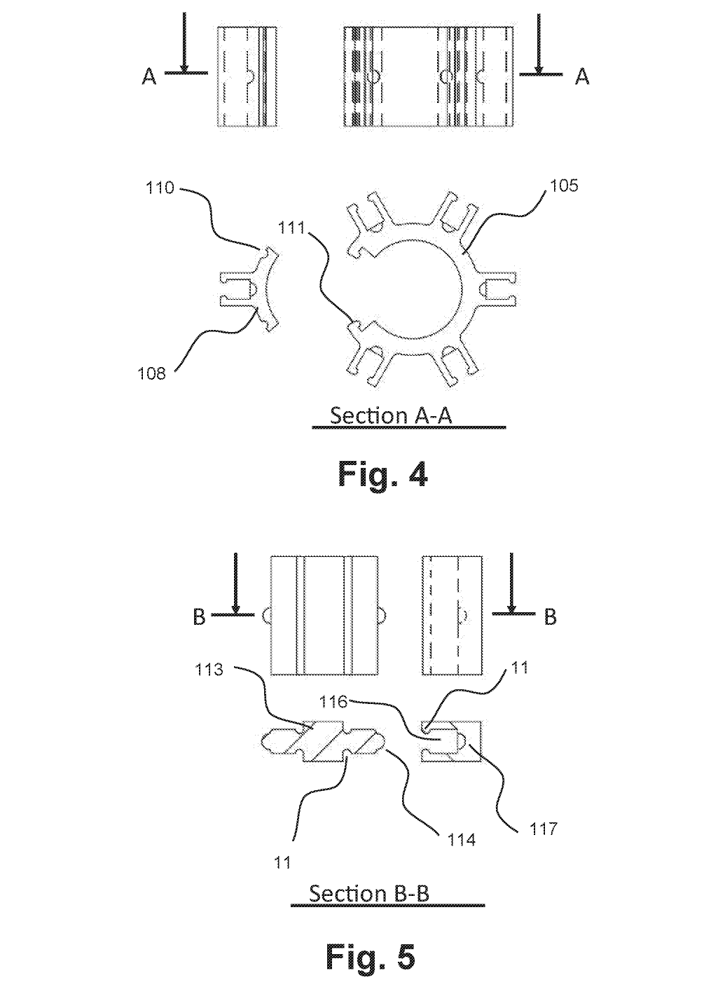

The semi-circular clip 105 has the same internal diameter as the outer diameter of the pylon 102. A semicircular opening 200 in the flexible clip allows it to be installed onto the pylon 102 with minimal force. The rear retaining clip 108 is used to secure the clip in place by sliding its hemispherical grooves FIG. 4, 110 into place FIG. 4, 111 on the semi-circular clip 105, mating the two pieces together. The rotation of the semi-circular clip 105 is stopped by a rotational braking pad 109 attached to the rear retaining clip 108. Female attachment receptors 201 are place at intervals around the semi-circular clip 105 and on the rear retaining clip 108.

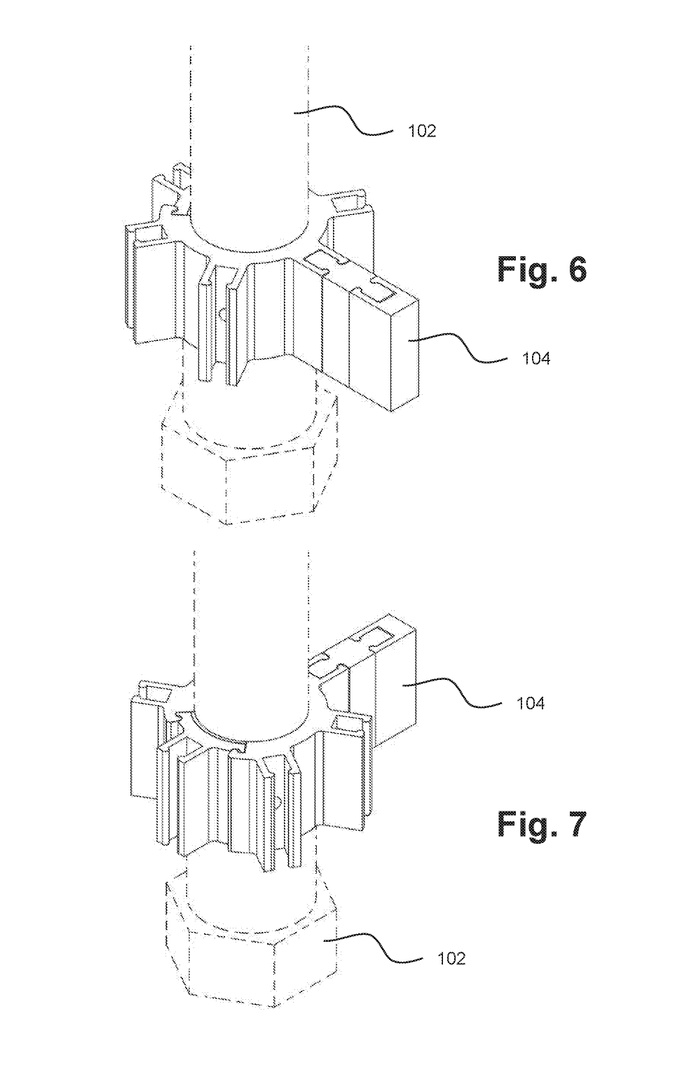

Double ended male bridge pieces 106 are snapped into the female attachment receptors 201 using hemispherical grooves FIG. 6, 112 mating the two pieces together, and a ball and socket system, FIGS. 5 114 and 117, to provide positive registration. The end clips 107 are snapped into the male bridge 106 using hemispherical grooves FIGS. 5 112 and 115, mating the two pieces together, and a ball and socket system FIGS. 5 114 and 117, to provide positive registration.

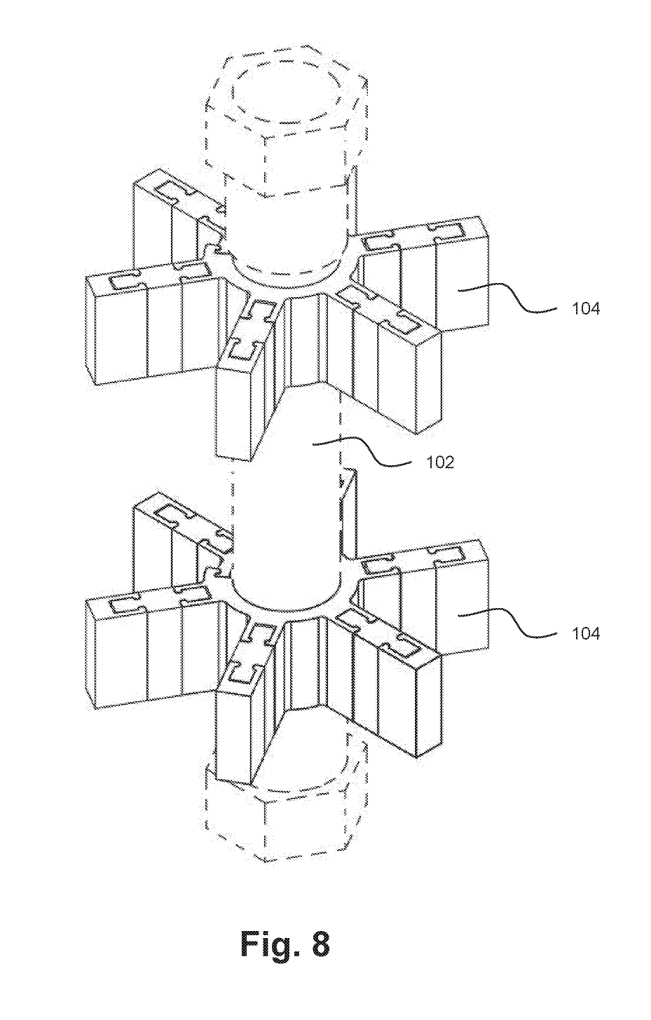

FIG. 6 and FIG. 7 illustrate the complete and installed assembly 104 on the existing pylon 102. FIG. 8 illustrates two fully populated assemblies 104 on an existing pylon 102.

The components are preferably formed by using a plurality of polyimide layers via an additive manufacturing process to insure flexibility and strength.

1. A prosthetic pylon attachment device, an apparatus for mechanically attaching accessories to a prosthetic pylon comprising:
a combination of a plurality of legs each comprising a far portion and a near portion, with each portion connecting to the other;
a central semi-circular portion with a plurality of radial members connecting to each of the plurality of leg portions;
a semi-circular rear retaining portion, connecting with the central portion, with a single radial member connecting to a single leg portion;
a semi-circular braking portion contacting to the least radius surface of the semi-circular rear retaining portion.

2. An embodiment of the apparatus of claim 1 wherein said plurality legs are of varying lengths.

3. An embodiment of the apparatus of claim 1 wherein said plurality of radial members are of varying quantity and position.

4. An apparatus of claim 1 wherein said connections utilize hemispherical ball and hemispherical socket connections to register connection.

5. An apparatus of claim 1 wherein said connections utilize hemispherical extrusion and hemispherical groove connections to register connection.

6. An apparatus of claim 1 wherein said leg, central and rear retaining portions are manufactured from polyimide material utilizing additive processes.
